(19) United States
(12) Patent Application Publication
(10) Pub. No.: US 2026/0295149 A1
(43) Pub. Date: Oct. 1, 2026

(54) DIRECT CONTROL OF TARGET-CONTROLLED INFUSION PROCEDURES AND OTHER MULTI-STEP INFUSION PROCEDURES

(71) Applicant: Fresenius Kabi USA, LLC, Lake Zurich, IL (US)

(72) Inventors: Benjamin G. Powers, Portsmouth, NH (US); Matthew B. Weinger, Nashville, TN (US)

(21) Appl. No.: 19/629,409

(22) Filed: Mar. 26, 2026

Related U.S. Application Data
(60) Provisional application No. 63/781,000, filed on Mar. 31, 2025.
(60) Provisional application No. 63/825,878, filed on Jun. 18, 2025.

(51) Int. Cl. A61M 5/142

(52) U.S. Cl. CPC A61M 5/142; A61M 2005/14208; A61M 2005/14296; A61M 2202/0007; A61M 2202/0241; A61M 2205/502

(57) ABSTRACT

A system for implementing a target-controlled infusion procedure or other multi-step infusion procedure includes a pump, an interface having a display screen, and a controller that is operatively coupled to the pump and the interface and programmed to receive data regarding a target-controlled infusion procedure or other multi-step infusion procedure to be executed. The controller controls the display screen to show a plurality of aspects of the procedure to be executed for a subject and controls the pump to execute the procedure according to the aspects shown on the display screen. The interface receives input from an operator regarding a change in at least one of the aspects of the procedure, with the controller receiving that input from the interface and controlling the display screen to show at least one changed aspect of the procedure, and with execution of the procedure being in accordance with the changed aspect.

### CROSS-REFERENCE TO RELATED APPLICATIONS

This application claims the benefit of and priority of U.S. Provisional Patent Application Ser. No. 63/781,000, filed Mar. 31, 2025, and U.S. Provisional Patent Application Ser. No. 63/825,878, filed Jun. 18, 2025, the contents of which are incorporated by reference herein.

### BACKGROUND

### Field of the Disclosure

The present subject matter relates to target-controlled infusion (“TCI”) systems and procedures and other multi-step infusion systems and procedures. More particularly, the present subject matter relates to systems and methods for directly controlling aspects of a TCI or other multi-step infusion procedure.

### Description of Related Art

TCI is an advanced drug delivery method designed to achieve and maintain a desired concentration of a drug in a specific compartment of the body, often referred to as the “effect site.” TCI utilizes pharmacokinetic and pharmacodynamic modeling to calculate the precise rates of drug infusion required to achieve a target therapeutic effect while minimizing side effects. A three-compartment model is commonly employed to characterize the distribution and elimination of drugs in the body. The three-compartment model (which is shown in FIG. 1) divides the body into the following compartments: a central compartment (which represents the blood plasma and organs with rapid drug equilibration, such as the heart and lungs) and two peripheral compartments. One of the peripheral compartments represents tissues that equilibrate more quickly (e.g., muscle), while the other peripheral compartment represents tissues with slower drug equilibration (e.g., fat). The effect site itself (which is not illustrated in FIG. 1) is not a physical compartment, but rather is the location where the drug exerts its pharmacodynamic effect, often associated with receptor interaction. For example, for drugs that primarily act on centers in the brain, the effect site is primarily the brain.

Drug movement between these compartments is governed by rates of distribution, metabolism, and elimination, which are defined by pharmacokinetic parameters. Drug movement from the central compartment to the first peripheral compartment is represented in FIG. 1 at “k₁₂” and drug movement from the first peripheral compartment to the central compartment is represented in FIG. 1 at “k₂₁”. Drug movement from the central compartment to the second peripheral compartment is represented in FIG. 1 at “k₁₃” and drug movement from the second peripheral compartment to the central compartment is represented in FIG. 1 at “k₃₁”. A TCI system continuously calculates infusion rates to maintain the desired concentration at the effect site based on real-time estimates of drug dynamics while taking into account pharmacokinetics.

A TCI infusion typically follows a well-defined sequence to achieve and maintain the target effect site concentration. The first step of the sequence is an initial bolus, in which a bolus dose is delivered at the start of the infusion to rapidly achieve the target concentration in the central compartment and facilitate drug distribution toward the effect site. Delivery of the bolus dose is represented in FIG. 1 at “I”.

Following the bolus, there may be a brief delay or significant reduction in infusion rate, which may be referred to as a “delay phase” of the sequence. This delay allows the drug to equilibrate between compartments and approach the target concentration in the effect site. Next, a sequence of infusion rates and durations is employed to fine-tune the drug concentration. These rates are dynamically adjusted by the TCI system to achieve steady-state conditions at the effect site. This may be referred to as the “titration phase” of the sequence.

Once the target effect site concentration is reached, the infusion rate stabilizes at a level sufficient to counterbalance the drug's clearance and distribution, maintaining the desired therapeutic effect. This may be referred to as “steady-state maintenance.” Eventually, the drug is eliminated from the body, with elimination of the drug from the central compartment being represented in FIG. 1 at “k₁₀”.

This structured approach minimizes fluctuations in drug concentration and ensures precise control over drug effects, making TCI particularly valuable for drugs with narrow therapeutic windows or complex pharmacokinetics. It is widely used in anesthesiology, critical care, and other clinical settings where precise dosing is essential.

Frequently, the target effect site concentration set by the clinician is varied over time. Examples might include increasing the level of sedation when a particularly intense portion of a procedure is about to be completed (such as spreading the chest in an open-heart procedure) or decreasing the level of sedation later in a procedure to reduce the effects of the drug and speed post-operation wakeup time.

In the current state of the art, these target changes are entered by a clinician at the time the change is desired and a controller of the TCI system utilizes the TCI model to calculate the required changes to the infusion rates (e.g., new bolus, delay, titration, and steady-state phases) based on the current effect site concentration and the new target set by the clinician; this controls the operation of the TCI system to implement the necessary change(s) to the infusion rate(s). This means that the clinician needs to remember to change the target before the new concentration is actually needed. In the case of a desired effect site increase, a failure to adjust the TCI settings enough in advance may cause a delay of that portion of the procedure that requires the new target until that target is achieved at the effect site. In the case of an anticipated need for a lower effect site concentration in the future, a delay in implementing the lower TCI settings could lead to delays in wake-up time or other undesirable side-effects.

Additionally, these changes are manually entered as new target concentrations, which does not provide the clinician with good contextual visual display of the time history of the effect site concentration levels or the delays expected between a target change and achievement of the desired effect site concentration based on the pharmacokinetics and pharmacodynamics (“PK/PD”) of the drug in the body. This is especially problematic for drugs that take a substantial amount of time for a rate change to impact the therapeutic effect of the drug-for example, hypnotic or opioid drugs used for anesthesia that are being controlled by a TCI system.

Accordingly, it would be advantageous to provide an approach whereby a visual display presents historic and predicted target and calculated effect site concentrations and enables a clinician to modify various aspects of a TCI procedure while more clearly understanding how their changes will impact patient response.

### SUMMARY

There are several aspects of the present subject matter which may be embodied separately or together in the devices and methods described and claimed below. These aspects may be employed alone or in combination with other aspects of the subject matter described herein, and the description of these aspects together is not intended to preclude the use of these aspects separately or the claiming of such aspects separately as set forth in the claims appended hereto.

In one aspect, a system for executing a target-controlled infusion procedure or other multi-step infusion procedure includes a pump, an interface having a display screen, and a controller that is operatively coupled to the pump and the interface and programmed to receive data regarding a target-controlled infusion procedure or other multi-step infusion procedure to be executed for a patient or other recipient of the intended infusion (henceforth, “subject”). The controller is also programmed to control the display screen to show a plurality of aspects of the infusion procedure to be executed for the subject and control the pump to execute the infusion procedure for the subject according to the aspects shown on the display screen. The interface is configured to receive input from an operator regarding a change in at least one of the aspects of the infusion procedure, with the controller being programmed to receive that input from the interface and control the display screen to show at least one changed aspect of the infusion procedure, with execution of the infusion procedure being in accordance with that changed aspect.

In another aspect, a controller-implemented method is provided for executing a target-controlled infusion procedure or other multi-step infusion procedure in which a drug is conveyed to a subject. The method includes receiving data regarding the target-controlled infusion procedure or other multi-step infusion procedure to be executed for the subject, controlling a display screen of an interface to show a plurality of aspects of the infusion procedure to be executed for the subject, and controlling a pump to execute the infusion procedure for the subject according to the aspects shown on the display screen. The interface is configured to receive input from an operator regarding a change in at least one of the aspects of the infusion procedure, with the controller being programmed to receive that input from the interface and control the display screen to show at least one changed aspect of the infusion procedure, with execution of the infusion procedure being in accordance with that changed aspect.

### BRIEF DESCRIPTION OF THE DRAWINGS

FIG. 1 is a diagrammatic view of a three-compartment model employed in a TCI procedure;

FIG. 2 is a front plan view of an exemplary embodiment of a system for executing a TCI procedure or other multi-step infusion procedure, according to aspects of the present disclosure; and

FIGS. 3-16 illustrate exemplary images appearing on a display screen of an interface of the system of FIG. 2, according to aspects of the present disclosure.

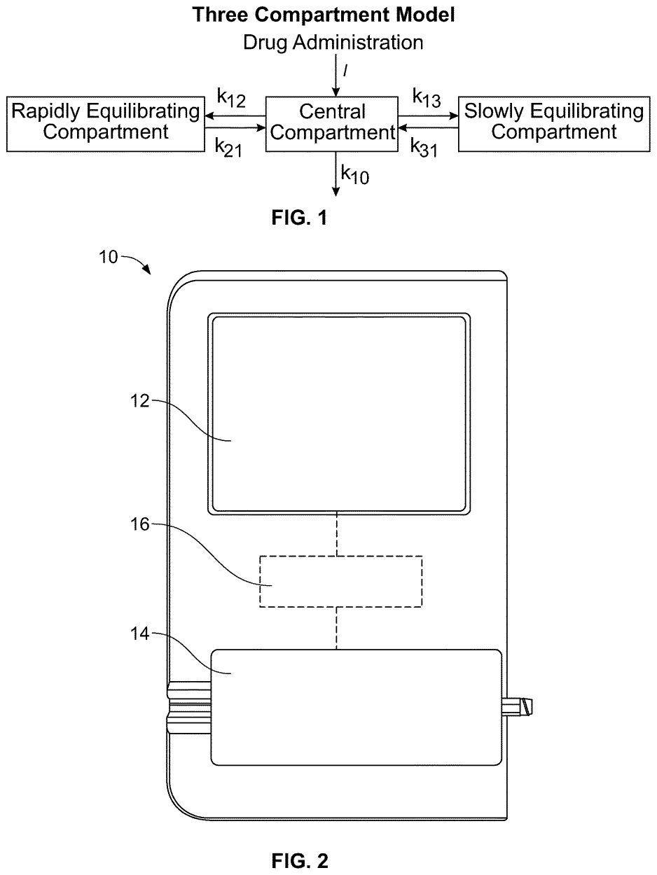

### DESCRIPTION OF THE ILLUSTRATED EMBODIMENTS

The embodiments disclosed herein are for the purpose of providing an exemplary description of the present subject matter. They are, however, only exemplary, and the present subject matter may be embodied in various forms. Therefore, specific details disclosed herein are not to be interpreted as limiting the subject matter as defined in the accompanying claims.

FIG. 2 illustrates an exemplary embodiment of an infusion system 10 embodying aspects of the present disclosure. In the illustrated embodiment, the infusion system 10 includes an interface 12, a pump 14, and a controller 16 that is operatively coupled to the interface 12 and the pump 14 and configured to control operation of each. The controller 16 may be variously configured, provided that it is capable of being programmed to implement the infusion procedure control principles described herein. While the controller 16 may be pre-programmed to implement these control principles, the instructions given to the controller 16 may be provided by a clinician or other human operator at the infusion system 10, by a clinician or other human operator from a remote location (e.g., from an authorized and secure dashboard, electronic medical record, or other remote control station), or by an appropriately authorized and secure autonomous system (e.g., artificial intelligence agent), for example. In one exemplary embodiment, the controller 16 comprises a main processing unit (MPU), which can comprise, e.g., a Pentium™ type microprocessor made by Intel Corporation, although other types of conventional microprocessors can be used. Similarly, the interface 12 and the pump 14 may be variously configured without departing from the scope of the present disclosure. In one embodiment, the interface 12 has one or more display screens with touch screen functionality, whereas such functionality may be omitted from the display screen(s) of the interface 12 in other embodiments, with input being provided to the interface 12 via a separate input device, such as a keyboard and/or a computer mouse, or by any other suitable means (e.g., via voice control).

It should be understood that the infusion system 10 of FIG. 2 is merely exemplary and that an infusion system according to the present disclosure may be differently configured without departing from the scope of the present disclosure. This may include the infusion system and individual components thereof being differently configured and/or oriented and/or the infusion system including additional components (e.g., one or more valves or alert lights or speakers or input devices). While FIG. 2 shows the interface 12, the pump 14, and the controller 16 incorporated into a single housing or device, it should be understood that two or more of these components of the infusion system 10 may be physically separate. For example, in one embodiment, the interface 12 may be incorporated into a desktop computer or a smart device (e.g., a tablet computer) or the like, with the interface 12 remotely communicating with the controller 16, which may itself be incorporated into the same device or housing as the pump 14 or into a separate device or housing. This may also include the infusion system 10 including a first controller that controls operation of the interface 12 and a second controller that communicates with the first controller and controls operation of the pump 14.

An infusion system 10 according to the present disclosure is capable of implementing “Target Controlled Infusion—Direct Control” (“TCI-DC”), which provides the ability to control the target effect site concentration (“ESC”) directly from the ESC interface 12. As will be described, in one embodiment, this may be a graphical “what you see is what you get” implementation of TCI-DC via a display screen of the interface 12. The controller 16 of the infusion system 10 may also be programmed to cause a display screen of the interface 12 to show historical, current, and projected levels of ESC over time. In one embodiment, TCI-DC further enables setting of future targets for the ESC to be staged over time (e.g., a future target or targets to be set) without changing the current target setpoint. Such future targets can be established at any time after the start of the infusion procedure and adjusted at any later time. This provides the ability to map out varying levels of a drug's effect (e.g., sedation) through a procedure and adjust them dynamically as the procedure progresses, all with clear visual contextual information to ensure clear understanding by the user (e.g., illustrating the effect of a particular bolus at a particular time on ESC).

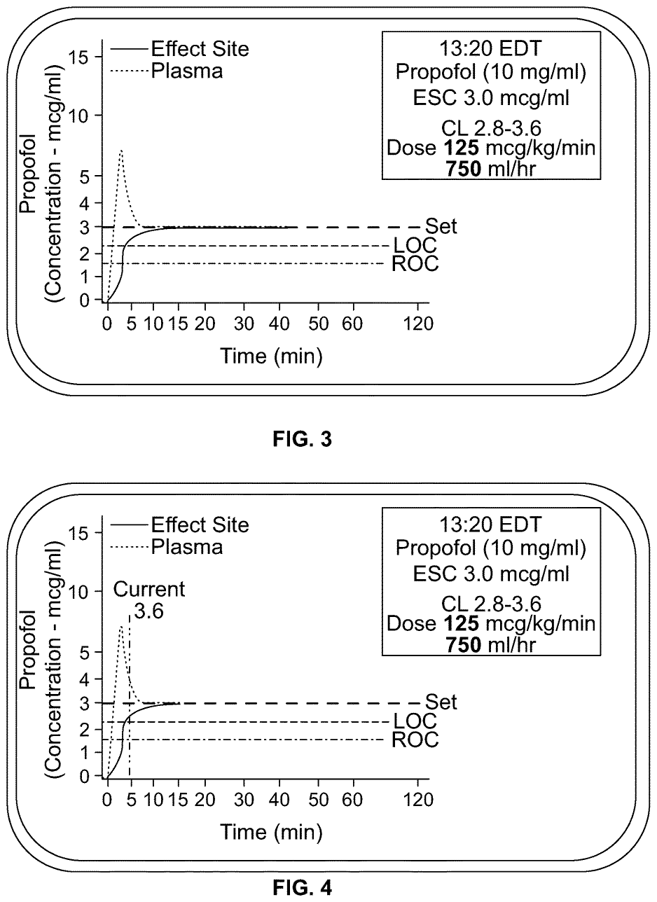

FIGS. 3-16 illustrate exemplary images that may be shown on a display screen of the interface 12 of the infusion system 10, with the images on the display screen being manipulated by a user to provide information and/or commands to the controller 16, which executes an infusion procedure based on input from the user. It should be understood that the illustrated images are merely exemplary and that images having different appearances and presenting different information (e.g., the volume of a drug remaining in a supply container or the time left before a patient is expected to regain consciousness) compared to those shown in FIGS. 3-16 are within the scope of the present disclosure. It should also be understood that the appearance of the images shown by the display screen may be adjusted by a user before or during a procedure, for example by selecting between the time axis of the graph showing minutes from the start of the infusion or showing actual clock time. Additionally, it should be understood that the images on the display screen may be manipulated by any suitable component of the interface 12 (e.g., a keyboard or computer mouse or voice control or using touch screen functionality of the display screen). Finally, it should be understood that, while the exemplary images show propofol as a representative drug (as it is a typical anesthetic agent that is commonly delivered using TCI), the same concepts described herein may be applied to any drug being administered using TCI or other multi-step infusion principles.

Prior to beginning an infusion procedure, the controller 16 of the infusion system 10 may be provided with various information and/or instructions regarding the procedure to be performed. This may include, for example, the controller 16 being informed of the drug to be delivered, appropriate patient demographic information needed for the TCI model (e.g., weight, age, sex, etc.), the target ESC, and input of any other patient-or procedure-or concurrent infusion drug-specific contextual information that would influence the safety or effectiveness of the drug being infused. The user may enter this data using the interface 12 or the data may be provided to the controller 16 in some other way, such as the controller 16 accessing an external database containing patient information or by the user operating a bar code scanner to read a bar code on a drug container. More generally, said information (and any commands to the controller that may follow) may be entered manually by one or more users of the infusion system at the location of the system, manually from a central station or other remotely located device (e.g., infusion dashboard), or automatically by querying the Electronic Health Record (EHR) or by an autonomous agent (e.g., artificial intelligence model running in the background).

When it has received the necessary information, the controller 16 calculates or otherwise determines various information regarding the procedure (e.g., the projected plasma and effect site concentrations expected over time once the infusion is started) and controls the display screen to show some or all of that information. This may include, for example, a visual indication of expected concentration, a numerical display of the drug, concentration, target ESC, dose-rate, and flow rate (FIG. 3). In the case of administration of a sedative, the display screen might also show the expected ESC levels for loss-of-consciousness (LOC) and return-of-consciousness (ROC) for the drug/patient combination. The various values could be set based on defaults (e.g., a default “deep sedation” ESC, a default “general anesthesia” ESC, a default “deep anesthesia” ESC, and a default “burst suppression” ESC), adjusted based on known patient attributes or physiology, entered by the clinician based on their knowledge/experience, adjusted based on observed effects during the procedure, or any combination of the above, with other factors also possibly being considered by the controller 16. Alternatively, dosing could be calculated and even automatically programmed (e.g., via an artificial intelligence model) based on general population data as well as previously measured specific hemodynamic level of consciousness, respiratory and other responses of the subject (patient or recipient) given extant ESC and other variables.

As the infusion is started and progresses, the display screen may show the historical trends of the concentration levels, the current ESC, and the projected levels going forward (FIG. 4). Various means can be used to indicate the difference between historical and projected values. In the example shown in FIGS. 3-16, the projected levels are lighter in tint and the historical values are bold. Other suitable means could be employed, such as use of a solid line for historical data versus a dashed line for projected (future) data, use of different colors or shapes, etc.

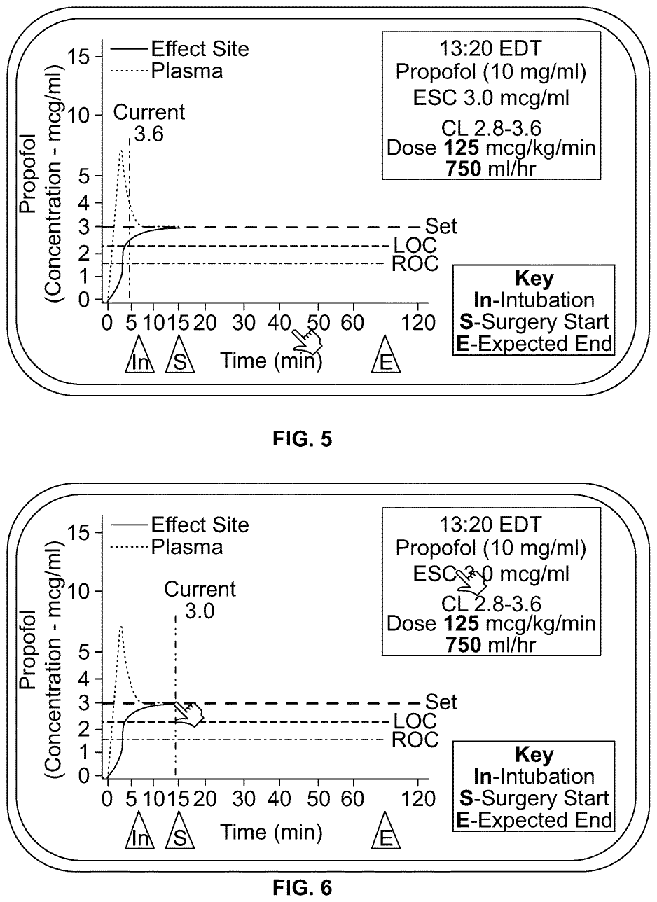

In one embodiment, the user is enabled to add flags for various events to the time axis shown on the display screen (FIG. 5), such as by pressing or clicking on the time axis. As the user interacts with the time axis, there could be a selection of typical flags (with FIG. 5 showing flags for intubation, the start of surgery, and an expected end) or the user could enter their own custom notation. In one embodiment, there could be different coloration for expected timing versus actual events that occurred (e.g., if surgery actually starts at a different time than what was entered at the beginning of the procedure). Alternatively (or additionally), event flags may be added to the display screen by the controller 16 in response to input retrieved by the controller 16 from any suitable source (e.g., from an external database of institutional or national guidelines or standards) and/or input received from an EHR system or any other suitable source (e.g., surgical scheduling software), which may include artificial intelligence-based decision support provided to the controller 16. This may also include the controller 16 including programming that causes it to add event flags to the display screen, which may include the controller 16 being programmed with artificial intelligence-based decision support and/or institutional or national guidelines or standards.

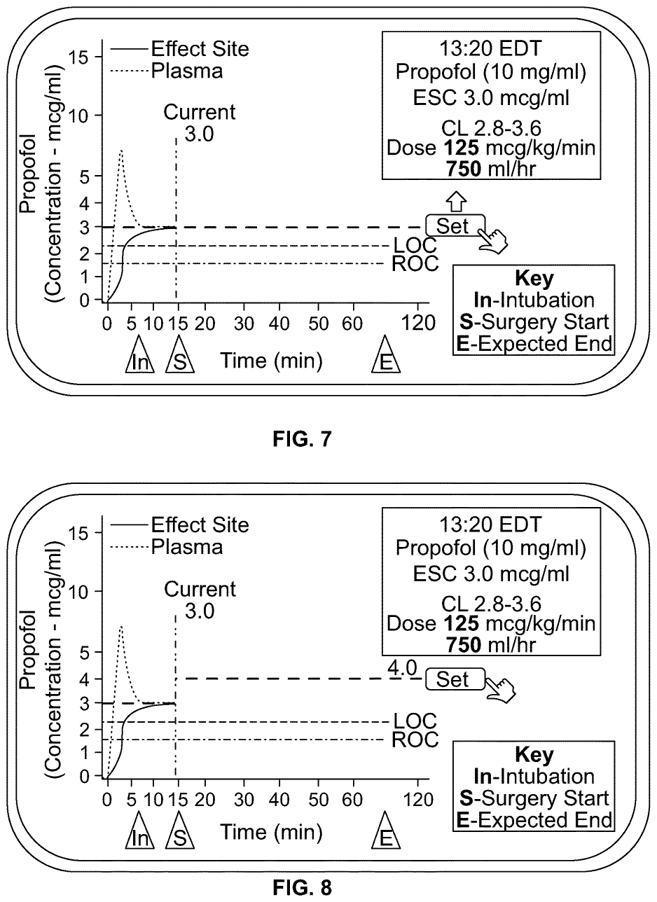

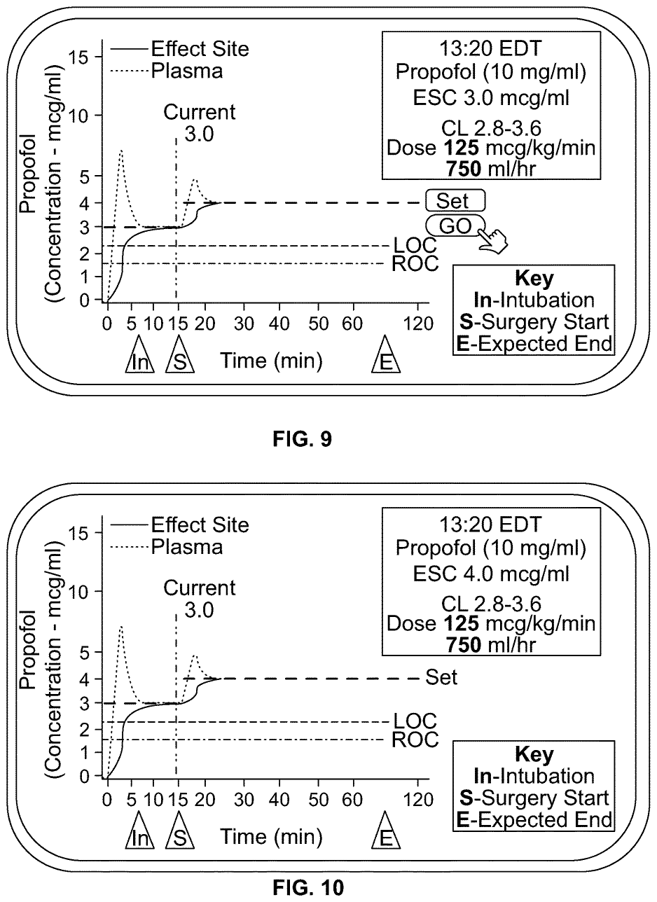

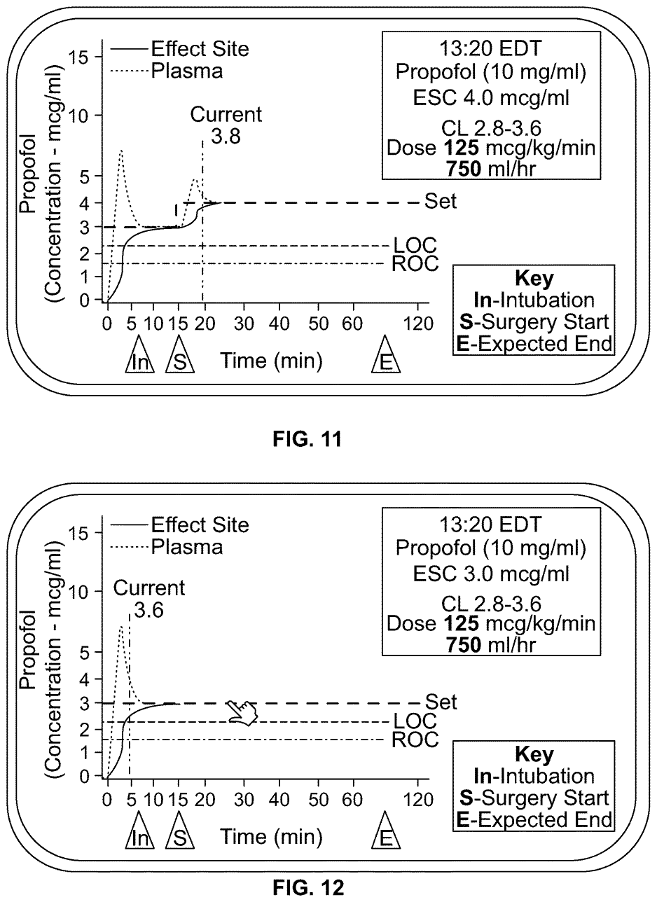

According to another aspect of the present disclosure, a user is enabled to change the target ESC using the display screen or some other component of the interface 12. For example, the user can click on or otherwise manipulate a numerical display of the ESC target or can click on and drag the setpoint in the ESC graph (FIG. 6). When directly manipulating the setpoint in the ESC graph, the user can drag the setpoint target up or down (with FIGS. 7 and 8 showing the setpoint being changed from a concentration of 3.0 mcg/ml to 4.0 mcg/ml), then manipulate a confirmation icon (with FIG. 9 showing a “go” button or icon) when the correct target is displayed, and the controller 16 of the system 10 will then control operation of the pump 14 so as to achieve the new target and update the current ESC value accordingly (with FIG. 10 showing the new setpoint having been set or confirmed and FIG. 11 showing ESC being increased so as to arrive at the new setpoint). The controller 16 may command the pump 14 to operate so as to achieve the different setpoint in any of a number of suitable ways, such as ramping up the rate at which a drug is being delivered to a patient or injecting a bolus (when moving to a higher setpoint) or ramping down the rate at which a drug is delivered to a patient or temporarily pausing drug delivery (when moving to a lower setpoint).

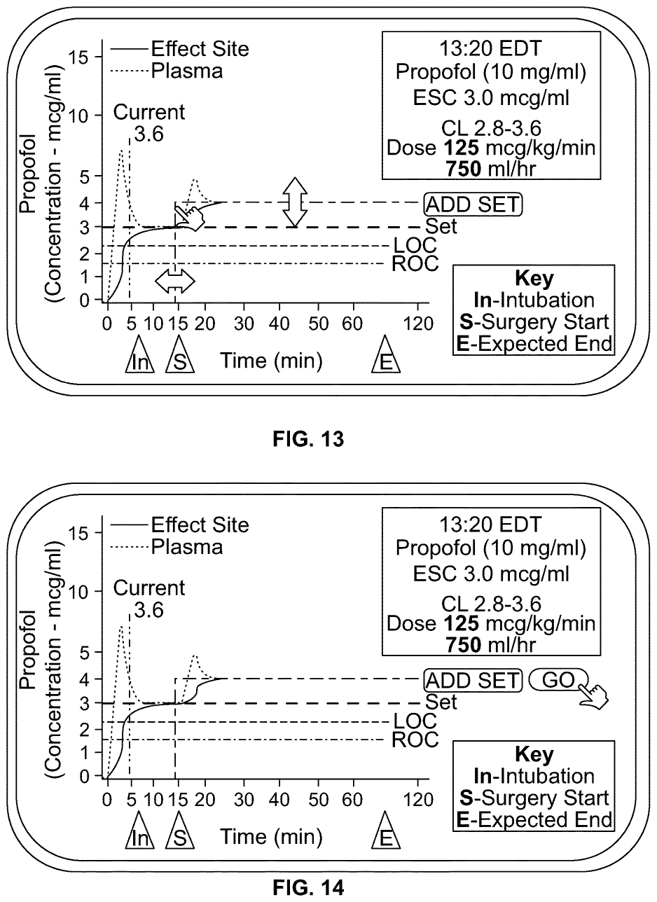

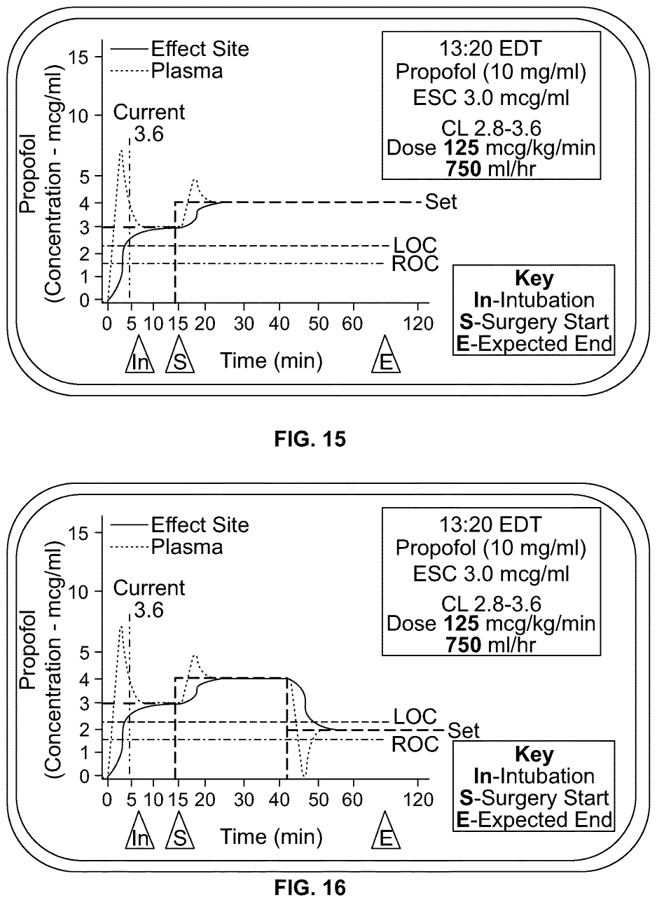

According to one embodiment, the user can also be enabled to directly control multiple changes to the target ESC over time using the display screen or some other component of the interface 12. This can occur either before the start of the infusion, during the infusion, or both. For example, a future location can be clicked on the graph (FIG. 12) and the target concentration and/or time of change of the target concentration can be changed by dragging around, with movement in a direction parallel to the time axis determining when a change is to be implemented and movement in a direction parallel to the concentration axis determining the nature of the change to be made (FIG. 13). FIG. 13 shows an example in which an operator has instructed the setpoint to be raised from 3.0 mcg/ml to 4.0 mcg/ml when surgery is scheduled to begin (fifteen minutes after a procedure has begun). The additional target can be confirmed in any suitable way, such as by an operator clicking on or otherwise manipulating a confirmation button or icon (FIG. 14) to confirm the time at which to make the change and the nature of the change. FIG. 15 shows the display screen after the change has been confirmed, with the color of the corresponding portion of the graph changing color, though it should be understood that some other change in the appearance of the graph may be employed to represent that a change has been confirmed. When the appropriate time in the infusion is reached, the target ESC will be changed as programmed, with the controller 16 adjusting the operation of one more pumps 14 of the system 10. If the procedure is progressing faster/slower than expected, the times and/or magnitudes of the ESC target changes can be adjusted by the user manipulating the display screen or some other component of the interface 12, such as by again clicking on the graph and dragging a portion thereof so as to instruct the controller 16 of the system 10 to cause an appropriate change in the operation of one or more pumps 14 of the system 10.

Multiple target setpoints can be added before and/or during a procedure, with FIG. 16 showing a first change (an increase) in target ESC that is to be implemented by the controller 16 at the 15-minute mark of the procedure (as selected by the operator in the steps illustrated in FIGS. 12-15) and a second change (a decrease) in target ESC that is to be implemented by the controller 16 after the forty-minute mark of the procedure. The second change in target ESC may be selected and implemented by an operator in accordance with the procedure described above and illustrated in FIGS. 12-15. The procedural flow shown in FIG. 16 illustrates target ESC increasing when surgery begins and then decreasing (to a level below LOC) some amount of time after surgery has begun, as may be appropriate in various surgical procedures.

It will be seen (by comparing FIG. 15 to FIG. 16) that implementation of the second change effectively changes the time at which the target ESC is to no longer be maintained at the level set per the first change and effectively changes the duration of the target ESC being maintained at the level set per the first change. It should be understood that the start and end time for maintaining the target ESC at a particular level may be changed using the display screen or some other component of the interface 12 without changing the duration of the target ESC being maintained at that level. For example, the operator may use the interface 12 to arrange for the controller 16 to execute a TCI procedure in which surgery is scheduled to begin at a particular time, with the target ESC to be kept at a particular level for thirty minutes after surgery has begun. If the time that surgery begins changes (e.g., if LOC occurs later than expected), the operator may use the interface 12 to make a corresponding change to the time at which the target ESC is to be set to the selected level without making a change to the duration of the target ESC being maintained at that level (e.g., by clicking on the thirty-minute portion of the graph and dragging the entire portion along the time axis to a different location).

While aspects of the foregoing description are specific to TCI procedures, it should be understood that the present disclosure is not limited to TCI procedures, but rather the principles described herein may also be employed when executing any other multi-step infusion procedure that includes a sequence of flow rates and durations over time.

### Aspects

Aspect 1. A system for executing a target-controlled infusion procedure or other multi-step infusion procedure, comprising: a pump; an interface including a display screen; and a controller operatively coupled to the pump and the interface and programmed to receive data regarding a target-controlled infusion procedure or other multi-step infusion procedure to be executed for a subject, control the display screen to show a plurality of aspects of the infusion procedure to be executed for the subject, and control the pump to execute the infusion procedure for the subject according to the aspects shown on the display screen, wherein the interface is configured to receive input from an operator regarding a change in at least one of said aspects of the infusion procedure, and the controller is programmed to receive said input from the interface and control the display screen to show at least one changed aspect of the infusion procedure, with execution of the infusion procedure being in accordance with said at least one changed aspect.
Aspect 2. The system of Aspect 1, wherein the interface is configured to receive said input before the pump is controlled by the controller to execute the infusion procedure, and the controller is programmed to control the pump to begin execution of the infusion procedure in accordance with said at least one changed aspect.
Aspect 3. The system of any one of the preceding Aspects, wherein the interface is configured to receive said input while the pump is being controlled by the controller to execute the infusion procedure, and the controller is programmed to change operation of the pump during execution of the infusion procedure in accordance with said at least one changed aspect.
Aspect 4. The system of any one of the preceding Aspects, wherein one of said aspects of the infusion procedure is a target effect site concentration of a drug conveyed to the subject during the infusion procedure, and the controller is programmed to receive input from the interface regarding a change in the target effect site concentration of the drug and to execute the infusion procedure in accordance with the changed target effect site concentration of the drug.
Aspect 5. The system of any one of the preceding Aspects, wherein one of said aspects of the infusion procedure is a time at which to convey a drug to the subject during the infusion procedure so as to achieve a target effect site concentration of the drug, and the controller is programmed to receive input from the interface regarding a change in the time at which to convey the drug to the subject during the infusion procedure so as to achieve the target effect site concentration of the drug and to execute the infusion procedure in accordance with the changed time at which to convey the drug to the subject so as to achieve the target effect site concentration of the drug.
Aspect 6. The system of any one of the preceding Aspects, wherein one of said aspects of the infusion procedure is a duration for which to maintain a target effect site concentration of a drug conveyed to the subject during the infusion procedure at a selected level, and the controller is programmed to receive input from the interface regarding a change in a time at which to convey the drug to the subject during the infusion procedure so as to achieve the target effect site concentration of the drug at said selected level without changing the duration for which to maintain the target effect site concentration of the drug at the selected level and to execute the infusion procedure in accordance with the changed time at which to convey the drug to the subject so as to achieve said selected level without changing the duration for which to maintain the target effect site concentration of the drug at the selected level.
Aspect 7. The system of any one of the preceding Aspects, wherein the interface is configured to receive input from the operator regarding an event during the infusion procedure to be shown on the display screen, and the controller is programmed to receive said input from the interface regarding said event and to control the display screen to show said event.
Aspect 8. The system of any one of the preceding Aspects, wherein the controller is programmed to control the display screen to show historic information regarding the infusion procedure during execution of the infusion procedure.
Aspect 9. The system of any one of the preceding Aspects, wherein the controller is programmed to control the display screen to show future information regarding the infusion procedure during execution of the infusion procedure.
Aspect 10. The system of any one of the preceding Aspects, wherein the interface is configured to receive said input before the pump is controlled by the controller to execute the infusion procedure and to receive a second input regarding a second change in at least one of said aspects of the infusion procedure while the pump is being controlled by the controller to execute the infusion procedure, and the controller is programmed to control the pump during execution of the infusion procedure in accordance with said input and said second input.
Aspect 11. The system of any one of the preceding Aspects, wherein the controller is configured to receive input from an external source regarding an event during the infusion procedure to be shown on the display screen, and the controller is programmed to control the display screen to show said event.
Aspect 12. The system of any one of the preceding Aspects, wherein the controller includes programming regarding an event during the infusion procedure to be shown on the display screen, and the controller is programmed to control the display screen to show said event.
Aspect 13. A controller-implemented method for executing a target-controlled infusion procedure or other multi-step infusion procedure in which a drug is conveyed to a subject, the method comprising: receiving data regarding the target-controlled infusion procedure or other multi-step infusion procedure to be executed for the subject; controlling a display screen of an interface to show a plurality of aspects of the infusion procedure to be executed for the subject; and controlling a pump to execute the infusion procedure for the subject according to the aspects shown on the display screen, wherein the interface is configured to receive input from an operator regarding a change in at least one of said aspects of the infusion procedure, and the controller is programmed to receive said input from the interface and control the display screen to show at least one changed aspect of the infusion procedure, with execution of the infusion procedure being in accordance with said at least one changed aspect.
Aspect 14. The method of Aspect 13, wherein the interface is configured to receive said input before the pump is controlled by the controller to execute the infusion procedure, and the controller is programmed to control the pump to begin execution of the infusion procedure in accordance with said at least one changed aspect.
Aspect 15. The method of any one of Aspects 13-14, wherein the interface is configured to receive said input while the pump is being controlled by the controller to execute the infusion procedure, and the controller is programmed to change operation of the pump during execution of the infusion procedure in accordance with said at least one changed aspect.
Aspect 16. The method of any one of Aspects 13-15, wherein one of said aspects of the infusion procedure is a target effect site concentration of the drug conveyed to the subject during the infusion procedure, and the controller is programmed to receive input from the interface regarding a change in the target effect site concentration of the drug and to execute the infusion procedure in accordance with the changed target effect site concentration of the drug.
Aspect 17. The method of any one of Aspects 13-16, wherein one of said aspects of the infusion procedure is a time at which to convey the drug to the subject during the infusion procedure so as to achieve a target effect site concentration of the drug, and the controller is programmed to receive input from the interface regarding a change in the time at which to convey the drug to the subject during the infusion procedure so as to achieve the target effect site concentration of the drug and to execute the infusion procedure in accordance with the changed time at which to convey the drug to the subject so as to achieve the target effect site concentration of the drug.
Aspect 18. The method of any one of Aspects 13-17, wherein one of said aspects of the infusion procedure is a duration for which to maintain a target effect site concentration of the drug conveyed to the subject during the infusion procedure at a selected level, and the controller is programmed to receive input from the interface regarding a change in a time at which to convey the drug to the subject during the infusion procedure so as to achieve the target effect site concentration of the drug at said selected level without changing the duration for which to maintain the target effect site concentration of the drug at the selected level and to execute the infusion procedure in accordance with the changed time at which to convey the drug to the subject so as to achieve said selected level without changing the duration for which to maintain the target effect site concentration of the drug at the selected level.
Aspect 19. The method of any one of Aspects 13-18, wherein the interface is configured to receive input from the operator regarding an event during the infusion procedure to be shown on the display screen, and the controller is programmed to receive said input from the interface regarding said event and to control the display screen to show said event.
Aspect 20. The method of any one of Aspects 13-19, wherein the controller is programmed to control the display screen to show historic information regarding the infusion procedure during execution of the infusion procedure.
Aspect 21. The method of any one of Aspects 13-20, wherein the controller is programmed to control the display screen to show future information regarding the infusion procedure during execution of the infusion procedure.
Aspect 22. The method of any one of Aspects 13-21, wherein the interface is configured to receive said input before the pump is controlled by the controller to execute the infusion procedure and to receive a second input regarding a second change in at least one of said aspects of the infusion procedure while the pump is being controlled by the controller to execute the infusion procedure, and the controller is programmed to control the pump during execution of the infusion procedure in accordance with said input and said second input.
Aspect 23. The method of any one of Aspects 13-22, wherein the controller is configured to receive input from an external source regarding an event during the infusion procedure to be shown on the display screen, and the controller is programmed to control the display screen to show said event.
Aspect 24. The method of any one of Aspects 13-23, wherein the controller includes programming regarding an event during the infusion procedure to be shown on the display screen, and the controller is programmed to control the display screen to show said event.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A system for executing a target-controlled infusion procedure or other multi-step infusion procedure, comprising:
a pump;
an interface including a display screen; and
a controller operatively coupled to the pump and the interface and programmed to
receive data regarding a target-controlled infusion procedure or other multi-step infusion procedure to be executed for a subject,
control the display screen to show a plurality of aspects of the infusion procedure to be executed for the subject, and
control the pump to execute the infusion procedure for the subject according to the aspects shown on the display screen, wherein
the interface is configured to receive input from an operator regarding a change in at least one of said aspects of the infusion procedure, and
the controller is programmed to receive said input from the interface and control the display screen to show at least one changed aspect of the infusion procedure, with execution of the infusion procedure being in accordance with said at least one changed aspect.

2. The system of claim 1, wherein
the interface is configured to receive said input before the pump is controlled by the controller to execute the infusion procedure, and
the controller is programmed to control the pump to begin execution of the infusion procedure in accordance with said at least one changed aspect.

3. The system of claim 1, wherein
the interface is configured to receive said input while the pump is being controlled by the controller to execute the infusion procedure, and
the controller is programmed to change operation of the pump during execution of the infusion procedure in accordance with said at least one changed aspect.

4. The system of claim 1, wherein
one of said aspects of the infusion procedure is a target effect site concentration of a drug conveyed to the subject during the infusion procedure, and
the controller is programmed to receive input from the interface regarding a change in the target effect site concentration of the drug and to execute the infusion procedure in accordance with the changed target effect site concentration of the drug.

5. The system of claim 1, wherein
one of said aspects of the infusion procedure is a time at which to convey a drug to the subject during the infusion procedure so as to achieve a target effect site concentration of the drug, and
the controller is programmed to receive input from the interface regarding a change in the time at which to convey the drug to the subject during the infusion procedure so as to achieve the target effect site concentration of the drug and to execute the infusion procedure in accordance with the changed time at which to convey the drug to the subject so as to achieve the target effect site concentration of the drug.

6. The system of claim 1, wherein
one of said aspects of the infusion procedure is a duration for which to maintain a target effect site concentration of a drug conveyed to the subject during the infusion procedure at a selected level, and
the controller is programmed to receive input from the interface regarding a change in a time at which to convey the drug to the subject during the infusion procedure so as to achieve the target effect site concentration of the drug at said selected level without changing the duration for which to maintain the target effect site concentration of the drug at the selected level and to execute the infusion procedure in accordance with the changed time at which to convey the drug to the subject so as to achieve said selected level without changing the duration for which to maintain the target effect site concentration of the drug at the selected level.

7. The system of claim 1, wherein
the interface is configured to receive input from the operator regarding an event during the infusion procedure to be shown on the display screen, and
the controller is programmed to receive said input from the interface regarding said event and to control the display screen to show said event.

8. The system of claim 1, wherein the controller is programmed to control the display screen to show historic information regarding the infusion procedure during execution of the infusion procedure.

9. The system of claim 1, wherein the controller is programmed to control the display screen to show future information regarding the infusion procedure during execution of the infusion procedure.

10. The system of claim 1, wherein
the interface is configured to receive said input before the pump is controlled by the controller to execute the infusion procedure and to receive a second input regarding a second change in at least one of said aspects of the infusion procedure while the pump is being controlled by the controller to execute the infusion procedure, and
the controller is programmed to control the pump during execution of the infusion procedure in accordance with said input and said second input.

11. The system of claim 1, wherein
the controller is configured to receive input from an external source regarding an event during the infusion procedure to be shown on the display screen, and
the controller is programmed to control the display screen to show said event.

12. The system of claim 1, wherein
the controller includes programming regarding an event during the infusion procedure to be shown on the display screen, and
the controller is programmed to control the display screen to show said event.

13. A controller-implemented method for executing a target-controlled infusion procedure or other multi-step infusion procedure in which a drug is conveyed to a subject, the method comprising:
receiving data regarding the target-controlled infusion procedure or other multi-step infusion procedure to be executed for the subject;
controlling a display screen of an interface to show a plurality of aspects of the infusion procedure to be executed for the subject; and
controlling a pump to execute the infusion procedure for the subject according to the aspects shown on the display screen, wherein
the interface is configured to receive input from an operator regarding a change in at least one of said aspects of the infusion procedure, and
the controller is programmed to receive said input from the interface and control the display screen to show at least one changed aspect of the infusion procedure, with execution of the infusion procedure being in accordance with said at least one changed aspect.

14. The method of claim 13, wherein
the interface is configured to receive said input before the pump is controlled by the controller to execute the infusion procedure, and
the controller is programmed to control the pump to begin execution of the infusion procedure in accordance with said at least one changed aspect.

15. The method of claim 13, wherein
the interface is configured to receive said input while the pump is being controlled by the controller to execute the infusion procedure, and
the controller is programmed to change operation of the pump during execution of the infusion procedure in accordance with said at least one changed aspect.

16. The method of claim 13, wherein
one of said aspects of the infusion procedure is a target effect site concentration of the drug conveyed to the subject during the infusion procedure, and
the controller is programmed to receive input from the interface regarding a change in the target effect site concentration of the drug and to execute the infusion procedure in accordance with the changed target effect site concentration of the drug.

17. The method of claim 13, wherein
one of said aspects of the infusion procedure is a time at which to convey the drug to the subject during the infusion procedure so as to achieve a target effect site concentration of the drug, and
the controller is programmed to receive input from the interface regarding a change in the time at which to convey the drug to the subject during the infusion procedure so as to achieve the target effect site concentration of the drug and to execute the infusion procedure in accordance with the changed time at which to convey the drug to the subject so as to achieve the target effect site concentration of the drug.

18. The method of claim 13, wherein
one of said aspects of the infusion procedure is a duration for which to maintain a target effect site concentration of the drug conveyed to the subject during the infusion procedure at a selected level, and
the controller is programmed to receive input from the interface regarding a change in a time at which to convey the drug to the subject during the infusion procedure so as to achieve the target effect site concentration of the drug at said selected level without changing the duration for which to maintain the target effect site concentration of the drug at the selected level and to execute the infusion procedure in accordance with the changed time at which to convey the drug to the subject so as to achieve said selected level without changing the duration for which to maintain the target effect site concentration of the drug at the selected level.

19. The method of claim 13, wherein
the interface is configured to receive input from the operator regarding an event during the infusion procedure to be shown on the display screen, and
the controller is programmed to receive said input from the interface regarding said event and to control the display screen to show said event.

20. The method of 13, wherein the controller is programmed to control the display screen to show historic information regarding the infusion procedure during execution of the infusion procedure.

21. The method of claim 13, wherein the controller is programmed to control the display screen to show future information regarding the infusion procedure during execution of the infusion procedure.

22. The method of claim 13, wherein
the interface is configured to receive said input before the pump is controlled by the controller to execute the infusion procedure and to receive a second input regarding a second change in at least one of said aspects of the infusion procedure while the pump is being controlled by the controller to execute the infusion procedure, and
the controller is programmed to control the pump during execution of the infusion procedure in accordance with said input and said second input.

23. The method of claim 13, wherein
the controller is configured to receive input from an external source regarding an event during the infusion procedure to be shown on the display screen, and
the controller is programmed to control the display screen to show said event.

24. The method of claim 13, wherein
the controller includes programming regarding an event during the infusion procedure to be shown on the display screen, and
the controller is programmed to control the display screen to show said event.
